(19) United States
(12) Patent Application Publication
(10) Pub. No.: US 2014/0199319 A1
(43) Pub. Date: Jul. 17, 2014

(54) METHODS FOR INCREASING THE EFFICIENCY OF HYBRIDOMA GENERATION

(71) Applicant: AbbVie, Inc., Worcester, MA (US)

(72) Inventors: Jane SEAGAL, Newton, MA (US); Eve H. BARLOW, Wellesley, MA (US); Chung-Ming HSIEH, Newton, MA (US); Jeffrey Yen PAN, Lake Forest, IL (US); Shawn M. JENNINGS, Acton, MA (US); Mary R. LEDDY, North Attleboro, MA (US); Archana B. THAKUR, Pleasanton, CA (US)

(73) Assignee: AbbVie, Inc., Worcester, MA (US)

(21) Appl. No.: 14/106,116

(22) Filed: Dec. 13, 2013

Related U.S. Application Data
(60) Provisional application No. 61/737,354, filed on Dec. 14, 2012.

(51) Int. Cl. C12N 5/16; G01N 33/569; C07K 16/00; C12N 15/02

(52) U.S. Cl. CPC C12N 5/163; C12N 15/02; G01N 33/56972; C07K 16/00
USPC 424/141.1; 435/449; 435/452; 435/450; 435/7.92; 435/7.1; 435/451; 435/355; 530/388.1; 435/325; 435/326; 506/9

(57) ABSTRACT

Disclosed herein are methods for improving the efficiency of the production of hybridomas, e.g., through enrichment of IgG expressing B cells. Also disclosed are hybridoma compositions produced using these methods as well as methods for producing antibodies with the hybridomas.

### BACKGROUND

Hybridoma technology is widely used for generating monoclonal antibodies for exploratory, diagnostic and therapeutic purposes. Hybrid cells (hybridomas) are produced by fusing ex vivo isolated cells from lymphoid organs or blood of the immunized species with tumor (myeloma) cells. The most commonly used technique to generate hybridomas from mouse splenocytes uses polyethylene glycol (PEG) as a fusogen (E Harlow, D. Lane, Antibodies: A Laboratory Manual, Cold Spring Harbor Laboratory Press, Cold Spring Harbor, N.Y., 1998) and is extremely inefficient, often producing inconsistent results. Improvements to the technique have the potential to increase both the throughput and the productivity of monoclonal antibody production.

Several factors contribute to the inefficiency of this procedure. Firstly, since the lymphoid organs of a mouse consist of multiple cell types and any of these cell types can fuse with a myeloma to generate a hybridoma line, many potential hybridomas have to be cultured and screened, to identify only a few that produce target specific IgG. Only hybridomas generated by the fusion of an IgG expressing B-lineage cell with a myeloma can produce IgG as a monoclonal antibody. In the B cell lineage, IgG expression is restricted mostly to post-germinal center B cells. This cell population, however, represents less than 5% of mouse lymphoid tissue. The remaining cell populations consist of non-B lineage (40-60%) and so called ‘naïve’ B cells that express surface IgM (40-60%). However, no efficient isolation of unmanipulated IgG expressing cells (including plasma cells) has been reported. Though several examples of sorting cells before fusion have been described in literature, all of them involve direct staining of these cells for surface markers followed by the application of the force of physical pressure to isolate relevant cell populations. This is known to negatively affect cell survival. Lastly, while electrofusion (e-fusion) has been reported to be much more efficient than PEG mediated fusion, the number of cells that can be used at any given time is limited. Multiple electrofusions must be set up to handle larger numbers of cells normally obtained from an immunized animal.

The present disclosure provides methods for increasing the efficiency of the hybridoma process thereby overcoming the deficiencies in the prior art.

### SUMMARY

The present disclosure provides methods for increasing the efficiency of hybridoma producing technology through enrichment of IgG expressing B cells.

The present disclosure provides a method of generating a hybridoma including the steps of providing murine spleen or lymph node cells and murine myeloma cells; isolating IgM negative B cells from the spleen or lymph node cells; and fusing the isolated IgM negative B cells and the murine myeloma cells, thereby generating hybridoma. In certain embodiments, the IgM negative B cells are isolated by immunodepletion of IgM positive cells and non-B cells.

In certain embodiments, murine spleen or lymph node cells are isolated from animals immunized with soluble recombinant proteins, cell lines, or purified human or humanized antibodies. In these embodiments, new hybridoma lines were generated using methods described in the present disclosure. These new hybridoma lines were producing antibodies specific for soluble recombinant proteins, cell-surface glycoproteins or anti-idiotypic antibodies recognizing variable domains of human or humanized antibodies.

In certain embodiments, the fusion can be performed by electrofusion. According to these embodiments, the method of generating a hybridoma also includes CpG stimulation of the isolated IgM negative B cells prior to fusion. The CpG stimulation can be performed for 1-24 hours prior to fusion. Specifically, the immunodepletion can be performed by depleting the murine spleen or lymph node cells of IgM positive cells. The immunodepletion can also be performed by depleting the murine spleen cells of one or more of TCR, CD4, CD90.2, CD11c, CD49b, Gr-1 or Ter-119 positive cells with or without depleting murine spleen or lymph node cells of IgM. The immunodepletion can also be performed by depleting the murine spleen or lymph node cells of non-B cells. The immunodepletion can also be performed by depleting the murine spleen or lymph node cells of IgM positive cells.

In another embodiment, fusion of the isolated IgM negative B cells and the murine myeloma cells is performed by electrofusion. In another embodiment, the method also includes the step of screening the hybridomas for antigen specific IgG. Specifically, the screening can be performed using ELISA, flow cytometry or imaging.

The present disclosure also provides a method of isolating IgM negative B cells from murine spleen or lymph node cells including the steps of providing murine spleen cells; and immunodepletion of IgM positive cells and non-B cells, thereby isolating IgM negative B cells. In certain embodiments, the IgM negative B cells are also TCR, CD4, CD90.2, CD11c, CD49b, Gr-1 or Ter-119 negative cells. The cells can also be substantially free of TCR, CD4, CD90.2, CD11c, CD49b, Gr-1 or Ter-119. In other embodiments, the method further includes the step of fusing the isolated IgM negative B cells and murine myeloma cells thereby generating hybridoma. Specifically, the method can further include the step of screening the hybridomas for antigen specific IgG.

The present disclosure also provides monoclonal antibodies produced by the methods disclosed herein. It also provides methods for using the monoclonal antibodies for the treatment of disease.

The present disclosure also provides a heterogeneous population of cells, wherein the cells are B cells and wherein the B cells are IgM negative. In certain embodiments, the B cells are TCR, CD4, CD90.2, CD11c, CD49b, Gr-1 or Ter-119 negative cells. The cells can also be substantially free of TCR, CD4, CD90.2, CD11c, CD49b, Gr-1 or Ter-119. In other embodiments, at least 25%, 50% or 75% of the B cells express IgG.

### BRIEF DESCRIPTION OF THE DRAWINGS

FIG. 1 is a schematic showing the principles of hybridoma technology.

FIG. 2 is a pie chart showing the relative abundance of lymphocyte populations in mouse spleen after conventional immunization.

FIG. 3 is a schematic showing sorting strategies to enrich for IgG expressing cells by positive sorting and by depletion of irrelevant cells.

FIGS. 4A and B are schematics showing PEG and electrofusion techniques, respectively.

FIG. 5 is a bar graph showing the reduction of cell number by pre-fusion depletion of IgM or CD43 expressing cells.

FIG. 6 is a bar graph showing decrease in hybridoma generation after depletion of CD43 but not IgM expressing cells.

FIG. 7 is a bar graph showing significant reduction in cell numbers using pre-fusion enrichment including combining B cell purification and depletion of IgM expressing cells.

FIG. 8 is a bar graph showing a lack of decrease in antigen specific hybridoma generation using pre-fusion enrichment of IgM negative B cells.

FIG. 9 is a bar graph showing increase in percent of positive hits using pre-fusion enrichment.

FIG. 10 is a bar graph showing that pre-fusion enrichment is compatible with the electro-fusion protocol.

FIG. 11A is a flow diagram showing experimental setup for data shown in FIGS. 11C and 11D.

FIG. 11B is a pie chart showing the relative abundance of lymphocyte populations in rat spleen.

FIG. 11C is a bar graph showing cell numbers using pre-fusion enrichment by combining depletion of T cells and IgM expressing B cells from rat spleen.

FIG. 11D is a bar graph showing a lack of decrease in antigen specific hybridoma generation using pre-fusion enrichment of IgM+ negative B cells by combined depletion of T cells and IgM expressing B cells from rat spleen.

FIG. 12 is a line graph showing size of mouse splenocytes after pre-fusion enrichment followed by CpG or non-CpG-ODN stimulation.

FIG. 13 is a bar graph showing increased fusion efficiency measured by total colony number, number of IgG producing colonies and number of antigen specific IgG producing colonies after CpG stimulation.

FIG. 14A is a bar graph showing comparable reduction in cell numbers using MACS or RoboSep systems for immunodepletion.

FIG. 14B is a bar graph showing comparable fusion efficiency for cells isolated using MACS or RoboSep systems.

FIG. 15A is a flow chart showing experimental setup for data shown in FIG. 15B

FIG. 15B is a bar graph showing comparable percent of cell recovery after depletion for cells isolated from spleen and from lymph nodes.

### DETAILED DESCRIPTION

In the present disclosure, methods are presented to optimize and evaluate cell sorting techniques to select specific B cell populations as a fusion partner to enhance the efficiency of the hybridoma generation process by decreasing the number of total colonies without deleterious effects on the number of target specific IgG producing hybridomas. Methods of the present disclosure test the suitability of sorted cells for electrofusion as determined by the total number of colonies and the percentage of hybridomas that produce target specific IgG. Hybridomas can be created by electrofusion methods or PEG-mediated fusion.

The disclosure herein demonstrates that a combination of pre-fusion cell enrichment provides a remarkable improvement in the efficiency of generating hybridomas producing target specific IgG. In particular, the method decreases the number of irrelevant hybridoma clones that must be grown and screened without decreasing the positive number of antigen binding monoclonal antibodies. This increases the capability of a single scientist to handle multiple fusions at the same time. Additionally, it facilitates the concurrent fusion of cells isolated from multiple organs or animals immunized with all classes of target antigen (soluble or cell bound) using different immunization protocols (adjuvants, routes of administration, frequency and number of boosts). This approach may be applied to cells isolated from any lymphoid organs or peripheral blood from different biological species.

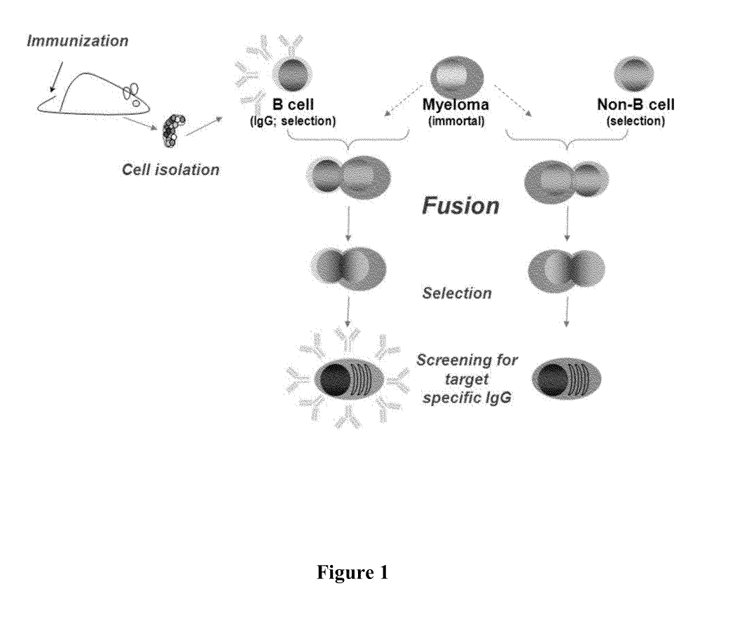

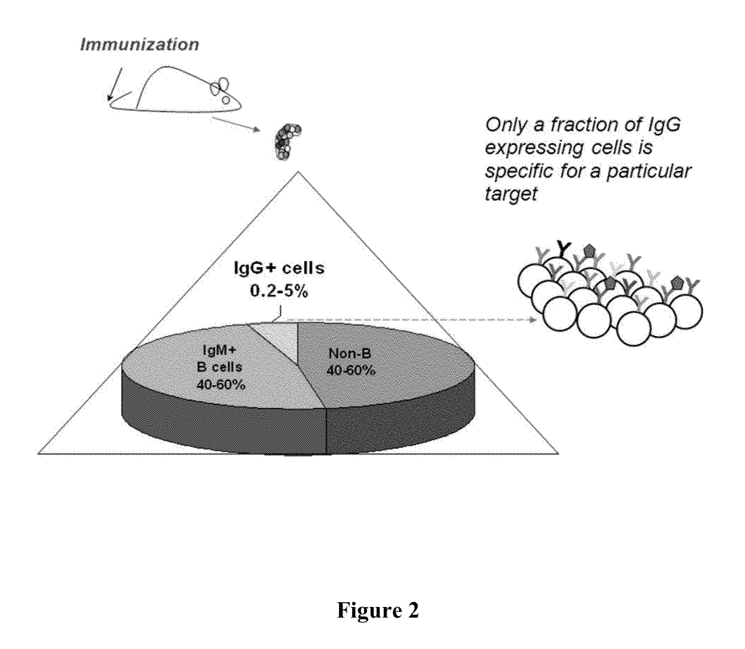

In certain embodiments, the current disclosure provides changes to the standard hybridoma techniques known in the art and summarized in FIG. 1. Embodiments of these techniques have been described in Kohler et al. ((1975) Nature 256:495), the human B-cell hybridoma technique (Kosbor et al. (1983) Immunology Today 4:72; Cole et al. (1983) Proc. Natl. Acad. Sci. USA 80:2026), and the EBV-hybridoma technique (Cole et al. (1983) Monoclonal Antibodies and Cancer Therapy, Alan R. Liss, Inc., pp. 77-96), incorporated herein by reference in their entireties. These techniques result in inefficiencies because the majority of splenic cells do not express IgG and thus are not appropriate for forming antibody generating hybridomas. Indeed, these techniques result in only a fraction of IgG expressing cells (e.g., 0.2-5%) that are specific for a particular target (see FIG. 2).

The current disclosure presents methods of isolating IgG producing B cells when making hybridomas. In certain embodiments, the cells from which the IgG producing B cells are isolated are from vertebrate splenocytes, for example rodent (e.g., mouse or rat) splenocytes. However, splenocytes can be isolated from any vertebrate animal amenable to hybridoma techniques, including for example, rabbits, goats, sheep, donkeys, llama, camels, monkeys, chimpanzees and humans. These splenocytes are isolated from any vertebrate animal to which are administered an antigen and optionally an adjuvant for the generation of antibodies within the animals. The isolation of IgG producing B cells is performed on the cells in vitro, but prior to fusion with myeloma cells producing a hybridoma. The methods described herein increase the efficiency of production of hybridomas that produce antibodies that specifically bind antigens that were administered to the animals from which the lymphoid cells are isolated. This is through the enrichment of B cells expressing IgG. These cells are enriched in B cells that express IgG that specifically bind to the antigen administered.

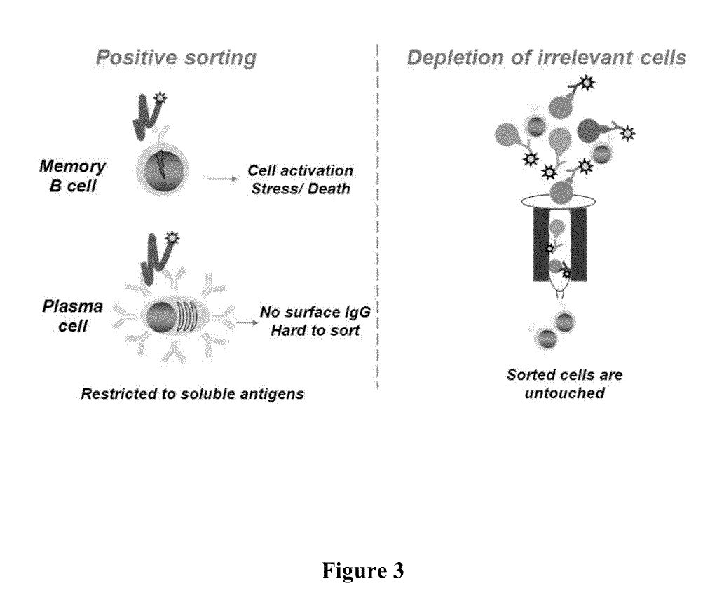

Pre-fusion cell sorting can be accomplished through positive sorting or depletion of irrelevant cells, summarized in FIG. 3. In positive sorting, cells can be isolated by direct staining with labeled antigen that can bind to the membrane bound IgG specific for a particular antigen. The disadvantage of this method is that B cells tend to activate when antigen binds to surface IgG, which may cause cell stress and often results in cell death. Further, plasma cells secrete IgG, but express very low (if any) levels IgG on their plasma membranes. Thus, positive sorting is unlikely to select these cells.

In certain embodiments, IgG producing B cell isolation is accomplished by immunodepletion of cells that are not IgG producing B cells. IgG producing B cells do not express IgM on their cell surfaces, while the vast majority non IgG producing B cells do express IgM on their cell surfaces. Thus, in one embodiment, the disclosure provides a method of increasing the efficiency of producing hybridomas that produce antigen specific IgG antibodies by immunodepletion of spleen cells from immunized mice using anti-IgM. This eliminates many if not substantially all of the IgM positive cells and enriches the remaining IgG producing B cells.

A pre-fusion cell sorting procedure was developed to significantly enrich for IgG expressing B cells by depleting inappropriate cells using antibodies to commonly known cell surface markers on non B-cell lineages and IgM expressing B cells. This approach led to a depletion of more than 90% of cells, which ultimately resulted in a smaller number of cells to fuse. Moreover, the isolated cells were minimally manipulated and therefore were well suited for productive hybridoma generation. This resulted in a reduction of the number of colonies to be cultured and screened and was without a deleterious effect on the number of target specific IgG expressing hybridoma lines obtained from the fusion.

Immunodepletion can be used with any antigen not expressed on IgG producing B cells. For instance antigens that are expressed on non-antigen producing B cells, T cells, myeloid lineage cells or red blood cells and not expressed on antibody producing B cells can be used to enrich antibody producing B cells. These antigens can include CD4 and CD90 which are expressed on T cells, CD11c, CD49b or Gr-1 which are expressed on myeloid lineage cells or Ter-119 expressed on red blood cells. These antigens can also include TCR, CD3, CD6 or CD8 expressed on T cells or NK1.1, CD14 or CD16 expressed on myeloid cells. Any of these antigens can be used for immunodepletion either alone or in any combination. These antigens can be used for immunodepletion in combination with IgM or without using IgM.

Immunodepletion can be performed using any method in the art. In one embodiment, antibodies specific for an antigen to be depleted are biotinylated. The antibodies are then introduced to the cells to be immunodepleted and anti-biotin or streptavidin bound microbeads are used to separate the antibodies and the cells they are bound to from the unbound cells. Immunodepleting antibodies can be linked to any antigen or tag to facilitate their isolation. These antigens can include FLAG epitopes, BSA or histidine tags. The antibodies can also be isolated using a secondary antibody that binds to any region of immunodepleting antibody. These regions include the Fc region, the heavy chain, light chain, Fv region or Fab region. The secondary antibody can be directly bound to a substrate or may itself be tagged as described above. In other embodiments, the immunodepletion of antibodies may be directly bound to a substrate. The substrate can include a bead (e.g. a microbead) or a surface, for example the surface of a well or a microplate. The separation can be done by flow cytometry or by magnetic field.

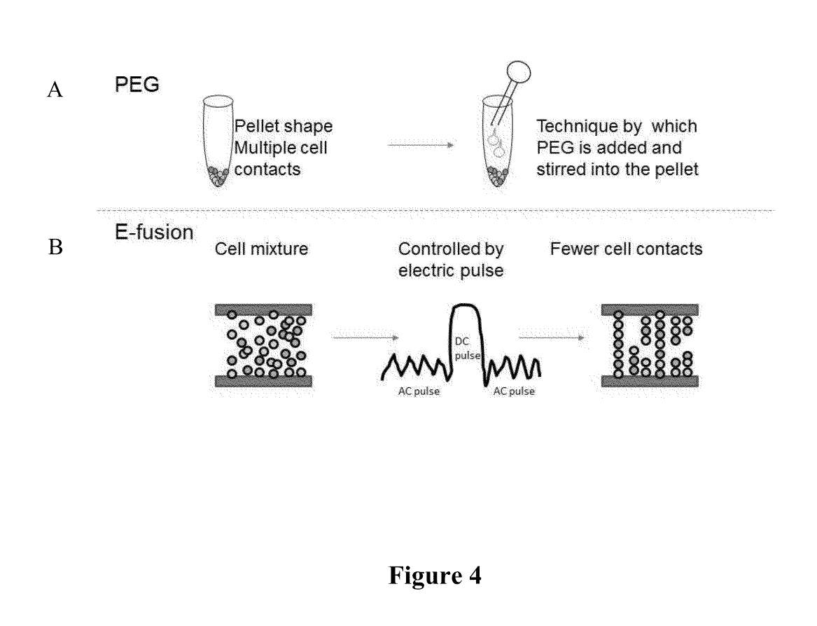

After immunodepletion, the remaining cells are subject to fusion with myeloma cells. Fusion can be performed according to any method known in the art. For example, electrofusion or PEG-mediated fusion may be used. These methods are summarized in FIGS. 4A and B. Typically, electrofusion is more efficient than the PEG-mediated fusion, but not suitable for large number of cells. However, using immunodepletion allows for the use of electrofusion by decreasing the number of cells that need to be fused.

Electrofusion can be further optimized through CpG stimulation of B cells prior to fusion. The optimal voltage for electrofusion inversely correlates with cell size. B cells and the myelomas with which they are fused differ in size. B cells are on average 7 μm in diameter, while myelomas are 12 μm in diameter. CpG stimulation can be used to increase the size of B cells and thus optimize the efficiency of electrofusion. CpG stimulation can be performed for 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 hours prior to electrofusion on sorted cells. These cells can be enriched in IgM negative B cells.

In certain non-limiting embodiments, splenocytes from rats can also be used in the methods of the invention to generate antibodies, especially in situations where cross-reactivity to mouse antigen is desired or when immune responses are hard to obtain in mice. Rat spleens contain 3-6×10⁸ cells. Thus, it is hard to handle conventional fusion outcomes from a single rat spleen due to the large number of clones. However, according to the methods disclosed herein, rat spleen is one of several viable alternatives as a source for splenic cells.

The inventive methods disclosed herein provide several advantages in various applications. Using classical fusion techniques, the majority of viable hybridoma clones do not produce IgG. Using the methods described herein, the pre-fusion sorting procedure allows the reduction of the number of clones that do not produce IgG as a result of fusion. Using previous hybridoma generation techniques, limited numbers of cells could be fused at the same time (e.g. cells from a single mouse spleen or part of rat spleen) due to significant workload that is required to culture and screen irrelevant viable colonies. Using the methods described herein, the sorted cells from several mouse spleens or full rat spleen could be fused in single fusion campaign without significant increase in the workload. Using the methods described herein, sorted splenic and lymph node cells can be fused at one time. Under previous fusion techniques, 5-10 fusions would be required to successfully complete a project. In certain exemplary embodiments, using the methods described herein, only 1-3 fusions would be required.

The cells remaining after immunodepletion include IgG expressing B cells. This heterogeneous cell population can contain 5-99% IgG expressing B cells. In other embodiments, this heterogeneous cell population can contain 10-99%, 20-99%, 30-99%, 40-99% or 50-99% IgG expressing B cells. In certain embodiments, it includes 5, 10, 15, 20, 25, 30, 35, 40, 45, 50, 55, 60, 65, 70, 75, 80, 85, 90, 95, 96, 97, 98 or 99% IgG expressing B cells. The heterogeneous cell population can include distinct subpopulations of B cells that express IgG that wherein the IgG from each subpopulation specifically binds to different antigens. This cell population can also be negative for CD4, CD90.2, CD11c, CD49b, Gr-1 or Ter-119. This cell population can also be negative for CD3, CD6, CD8, NK1.1, CD14 or CD16.

### DEFINITIONS

The term “antibody”, as used herein, includes monoclonal antibodies (including full length monoclonal antibodies), polyclonal antibodies, multispecific antibodies (e.g., bispecific antibodies), chimeric antibodies, complementarity determining regions (CDR) grafted antibodies, humanized antibodies, human antibodies and antigen-binding fragments thereof, for example, an antibody light chain (V_{L}), an antibody heavy chain (V_{H}), a single chain antibody (scFv), a F(ab′)₂ fragment, a Fab fragment, an Fd fragment, an Fv fragment, and a single domain antibody fragment (DAb). The term “immunoglobulin” may be used synonymously with antibody.

The term “chimeric antibody” is used to describe a protein comprising at least an antigen-binding portion of an immunoglobulin molecule that is attached by, for example, a peptide bond or peptide linker, to a heterologous protein or a peptide thereof. The “heterologous” protein can be a non-immunoglobulin or a portion of an immunoglobulin of a different species, class or subclass.

The term “isolated antibody”, as used herein, includes an antibody that has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies.

The term “antigen”, as used herein, includes an entity (e.g., a proteinaceous entity or peptide) to which an antibody specifically binds, and includes, e.g., a predetermined antigen to which both a parent antibody and modified antibody as herein defined bind. The target antigen may be polypeptide, carbohydrate, nucleic acid, lipid, hapten, or other naturally occurring or synthetic compound. Preferably, the target antigen is a polypeptide.

The term “immunogen”, as used herein, includes entity that can be used to elicit antigen-specific immune response in vivo, e.g. be polypeptide, carbohydrate, nucleic acid, lipid, hapten, or other naturally occurring or synthetic compound.

The term, “antigen-specific” as used herein refers to an interaction between the CDR regions of the immunoglobulin molecule with an epitope of the antigen wherein the CDR regions of the immunoglobulin molecule binds to the epitope.

The terms “cell”, “cell line”, “cell culture”, or “host cell”, as used herein, includes “transformants”, “transformed cells”, or “transfected cells” and progeny thereof. Host cells within the scope of the disclosure include prokaryotic cells such as E. coli, lower eukaryotic cells such as yeast cells, insect cells, and higher eukaryotic cells such as vertebrate cells, for example, mammalian cells, e.g., Chinese hamster ovary cells and myeloma cells.

The term “B cell”, as used herein, means an immature B cell, a mature naive B cell, a mature activated B cell, a memory B cell, a B lineage lymphocyte, plasmablast, plasma cell or any other B lineage cell of human origin or from non-human animal sources such as mice, for example.

The term “hybridoma” as used herein, refers to a cell or a cell line that is produced by fusing an antibody producing cell, e.g. a B cell, and an immortalized cell, e.g. a myeloma cell.

The term “screening”, as used herein, refers to an assay to assess the genotype or phenotype of a cell or cell product including, but not limited to nucleic acid sequence, protein sequence, protein function (e.g., binding, enzymatic activity, blocking activity, cross-blocking activity, neutralization activity, and the like). The assays include ELISA-based assays, Biacore analysis, and the like.

The term “Immunoglobulin G” or “IgG” as used herein, are antibody molecules each composed of four peptide chains, two heavy chains and two light chains. Each IgG has two antigen binding sites. Other immunoglobulins, e.g., IgM, may be described in terms of polymers with the IgG structure considered the monomer. IgG molecules are synthesized and secreted by plasma B cells. IgG antibodies are molecules of about 150 kDa composed of four peptide chains. They contain two identical heavy chains of about 50 kDa and two identical light chains of about 25 kDa, thus they have a tetrameric quaternary structure. The two heavy chains are linked to each other and to a light chain each by disulfide bonds. The resulting tetramer has two identical halves, which together form the Y-like shape. Each end of the fork contains an identical antigen binding site. There are four IgG subclasses (IgG1, 2, 3, and 4).

The term “Immunoglobulin M” or “IgM” as used herein, refers to an antibody that is produced by B cells and is comprised of a pentamer or hexamer of IgG subunits as well as other linking structures. It has a molecular mass of approximately 900 kDa in its pentamer form.

The term “cluster of differentiation” or “CD” as used herein, refers to a protocol used for the identification and investigation of cell surface molecules present on white blood cells. CD nomenclature provides targets for immunophenotyping of cells. CD molecules often act as receptors or ligands (the molecule that activates a receptor) important to a cell. The CD nomenclature system is commonly used as cell markers in immunophenotyping, allowing cells to be defined based on what molecules are present on their surface. These markers are often used to associate cells with certain immune functions.

The term “specifically binds,” means that an antibody or antigen-binding fragment thereof forms a complex with an antigen that is relatively stable under physiologic conditions. Specific binding can be characterized by a dissociation constant of at least about 1×10⁻⁶ M or smaller. In other embodiments, the dissociation constant is at least about 1×10⁻⁷ M, 1×10⁻⁸ M, or 1×10⁻⁹ M. Methods for determining whether two molecules specifically bind are well known in the art and include, for example, equilibrium dialysis, surface plasmon resonance, and the like.

The term “positive”, as used herein, refers to the expression of an mRNA or protein in a cell, wherein the expression is at least 5 percent of the expression of actin in the cell.

The term “negative”, as used herein, refers to the expression of an mRNA or protein in a cell, wherein the expression is less than 1 percent of the expression of actin in the cell.

### ABBREVIATIONS

### Ab—Antibody

### CD—Cluster of Differentiation

### ECM—Electro-Cell Manipulator

EFB—Electrofusion buffer
E—fusion-Electrofusion

### ELISA—Enzyme Linked Immunosorbent Assay

### FCS—Fetal Calf Serum

### GM-CSF—Granulocyte Macrophage Colony Stimulating Factor

### HA—Hypoxanthine-Azaserine

### HRP—Horseradish Peroxidase

### IgG—Immunoglobulin Gamma

### IgM—Immunoglobulin Mu

### IV—Intravenous

kDa—Kilo-Dalton

### MAb—Monoclonal Antibody

### MACS®—Magnetic Cell Separation

### PEG—Polyethylene Glycol

### PBS—Phosphate Buffered Saline

RT—Room temperature

### SA—Streptavidin

### SC—Subcutaneous

### SN—Supernatant

### TCR—T Cell Receptor

### TMB—3,3′,5,5′-Tetramethylbenzidine

### EXAMPLES

### Materials and Methods

### Cell Fusion Preparations

Murine myeloma cell lines were cultured to reach the log phase stage right before fusion. Cells were harvested from culture and counted. Spleen and lymph node cells from immunized animals were prepared sterilely, counted and sorted.

### Cell Sorting

Cells Isolated from Mouse Spleen Using MACS System (Miltenyi Biotec)

IgM expressing B cells were depleted with anti-mouse IgM microbeads. Anti-mouse CD43 microbeads or a mouse Pan B cell isolation kit was used to deplete non-B cells. All sorting procedures were performed on LS columns according to the manufacturer's protocol. To enrich for IgM negative B cells, splenocytes were first stained with a biotinylated-antibody cocktail (a component of the mouse Pan B cell isolation kit), then washed and incubated with anti-biotin and anti-mouse IgM microbeads. Cell depletion was performed using an LS column according to the manufacturer's protocol.

Cells Isolated from Mouse Spleen Using RoboSep System (StemCell Technologies)

A mixture of biotinylated anti-mouse IgM and a biotinylated-antibody cocktail (a component of the Mouse B cell enrichment kit) were used to enrich for IgG expressing B cells. For this procedure the biotinylated anti-mouse IgM was added directly to the splenocytes and the reagents from Mouse B cell enrichment kit (StemCell Technologies) were placed into RoboSep system. Cell depletion was performed using fully automated RoboSep system according to the manufacturer's protocol.

Cells Isolated from Rat Spleen

Rat splenocytes were first stained with biotinylated anti-Rat IgM and anti-Rat TCR antibodies, then washed and incubated with anti-biotin microbeads. The sorting procedure was performed on LS columns according to the manufacturer's protocol.

### PEG-Mediated Fusion:

Purified cells were fused with myeloma cells according to routine methods. Desired numbers of total splenocytes or sorted cells were combined with myeloma cells at ratios ranging from about 2:1 to about 10:1 and washed using serum free media and centrifuged for 10 minutes at 1000 rpm. The media was carefully aspirated from above the pellet and 1 mL of the PEG solution was slowly added and stirred into the cell pellet over 1 minute. The cell suspension was stirred with pipette for 1 more minute and the PEG solution was gradually diluted with the serum free media by adding 1 mL over 1 min, 3 mL over 1 minute, and 16 mL over 3 minutes. Fused cells were incubated at 37° C. for 5 min, centrifuged for 10 min at 1000 rpm, and washed with RPMI/10% FCS. Finally, fused, hybrid cells were resuspended in RPMI/20% FCS/HA selection media at a final concentration of approximately 2×10⁵ of myeloma cells/mL, dispensed into 96-well plates and cultured at 37° C., in medium 5% CO2.

### Electrofusion

Desired numbers of total splenocytes or sorted cells were combined with myeloma cells at ratios ranging from about 2:1 to about 10:1 and washed twice with electro-fusion buffer. Cell pellets were gently resuspended to 1.7×10⁶ cell/mL in EFB. Microslides were loaded with 0.6 mL of the cell suspension and micrograbber cables were attached to posts in the microslide chamber. The electro-fusion was performed using the following ECM set up:

Alignment amplitude: 75 V

Alignment time: 30 sec

Compression: 5 sec

Square pulse: 30 μsec

Electroporation amplitude: 640 V

Number of pulses: 1

Fused cells were transferred into 50 mL HA media, incubated for 1 hour at 37 C and plated into 96 well plates.

### Hybridoma Maintenance

Surviving hybridoma colonies were observed microscopically seven to ten days post-fusion and counted. Two weeks post-fusion, the supernatant from each well was screened by capture ELISA to determine the number of IgG producing clones and/or the number of clones producing antigen specific IgG. Hybridoma clones producing antibodies that showed high specific binding activity were subcloned, purified, and submitted for further analysis.

### Screening

### Capture ELISA

ELISA plates were coated with 50 μL of 2.0 μg/mL Goat Anti-Mouse IgG Fc in carbonate buffer (pH 9.4) overnight at 4° C. and washed 1× with PBS, 0.05% Tween-20. Wells were blocked with PBS, 2% Non-Fat Dry Milk for one hour at RT. Plates were washed three times after blocking. Hybridoma SNs were diluted 1:3 in PBS, 0.1% BSA and were added to each well (50 μL/well). Plates were incubated at RT for one hour. Wells were washed three times.

### Screening for IgG Producing Clones by Capture ELISA

To screen for IgG production, 50 μL of HRP conjugated Goat anti-mouse IgG Fc specific (1:10000) were added to each well and incubated for 1 hour at RT. After three washes, the plates were developed with TMB substrate. Hydrochloric acid was used to stop the reaction. Absorbance was read at 450 nm using a Molecular Device Spectramax 340.

### Screening for Antigen-Specific IgG Producing Clones by Capture ELISA

To screen for antigen specific IgG, 50 μL/well of biotinylated antigen at a concentration of 20-50 ng/mL was added and incubated for one hour at RT. Wells were washed three times.

SA-HRP was diluted 1:10000 in PBS, 0.1% BSA (50 μL/well), and incubated at room temperature for one hour. Wells were washed three times. TMB was added at 50 μL per well. Color development was stopped with hydrochloric acid (50 μL/well) Absorbance was read at 450 nm using a Molecular Device Spectramax 340.

### Screening for Antigen-Specific IgG Producing Clones by Direct ELISA

ELISA plates were coated with 50 μL/well of 2.0 μg/mL antigen in PBS buffer at pH 7.4 overnight at 4° C. and washed 1× with PBS, 0.05% Tween-20. Wells were blocked with PBS, 2% non-fat dry milk for one hour at RT. Plates were then washed three times after blocking. Hybridoma SNs were diluted 1:3 in PBS, 0.1% BSA, and then 50 μL were added to each well. Plates were incubated at RT for one hour. Wells were washed three times. 50 μL of HRP conjugated Goat anti-mouse IgG Fc specific (1:10000) were added to each well and incubated for 1 hour at RT. After three washes, the plates were developed with TMB substrate. Hydrochloric acid (50 μL/well) was used to stop the reaction. Absorbance was read at 450 nm using a Molecular Device Spectramax 340.

### Animal Strains

Mouse strains (A/J, Balb/c) were purchased from The Jackson Laboratory (Bar Harbor, Me.). HSD rats were purchased from Harlan (Indianapolis, Ind.). All animals were housed in the ABC animal facility. Immunizations were performed following IACUC protocol #21.

### Antigens

Recombinant soluble proteins, human monoclonal antibody, humanized monoclonal antibody, cell line expressing membrane glycoprotein.

### Cell Sorting Reagents and Equipment

See Table 1

### Cell Sorting Buffer

3% FCS in PBS

### Cell Lines

Murine myeloma cell lines: SP2/0-Ag14, ATCC CRL-1581, NS0 Medical Research Council

### Cell Culture and Fusion Reagents and Equipment

See Table 1

TABLE 1

| | | |
|---|---|---|
| | | Vendor Catalog |
| Name | Vendor | Number |

Cell sorting reagents and equipment

| | | |
|---|---|---|
| Anti-Mouse CD43 Microbeads | Miltenyi Biotec | 130-049-801 |
| Pan B cell isolation kit (Mouse) | Miltenyi Biotec | 130-095-813 |
| Anti-Mouse IgM Microbeads | Miltenyi Biotec | 130-047-302 |
| LS columns | Miltenyi Biotec | 130-042-401 |
| MACS MultiStand | Miltenyi Biotec | 130-042-303 |
| MidiMacs Separator | Miltenyi Biotec | 130-042-392 |
| Biotinylated anti-Rat IgM | BioLegend | 408903 |
| Biotinylated anti-Rat TCR | eBiosciences | 13-5960-85 |
| Anti-Biotin Microbeads | Miltenyi Biotec | 130-090-485 |
| Biotinylated Anti-Mouse IgM | BioLegend | 406504 |
| Mouse B cell Enrichment Kit | StemCell Technologies | Custom order |
| RoboSep ™ | StemCell Technologies | 20000 |

Cell culture and fusion reagents

| | | |
|---|---|---|
| Azaserine | Sigma | A-1164 |
| FCS | HyClone | SH30080 |
| Hybridoma Cloning Factor | PAA | F05-009 |
| PEG 1500 | Roche | 10 783 641 003 |
| Hypoxanthine | Calbiochem | 4010 |
| RPMI | Invitrogen | 11875 |
| Mannitol | Sigma | M8429 |
| MgSO₄ | Sigma | M7774 |
| CaCl₂ | Fisher Scientific | C79-500 |

Screening reagents:

| | | |
|---|---|---|
| Goat anti-Mouse IgG (Fc) | Jackson | 112-005-071 |
| | Immunoresearch | |
| HRP conjugated Goat anti- | Thermo Scientific | 31439 |
| Mouse IgG (Fc) | | |
| SA-HRP | Thermo Scientific | 21126 |
| TMB | Invitrogen | 00-203 |
| HCl | VWR | 3202-1 |
| Spectramax | Molecular Devices | Model 340 |

### E-Fusion Buffer (EFB)

0.3 M Mannitol

0.1 mM MgSO₄

0.1 mM CaCl₂, filter sterilized.

### Electro-Fusion Equipment

Electro Cell Manipulator ECM 2001 (Harvard Apparatus)

### Microslide

Microslides were used as follows: 3.2 mm gap size, maximum volume 0.7 mL, not autoclavable, rinse with water, followed by 70% ethanol and air dry in a tissue culture hood before each use (Harvard Apparatus, BTX model 453, cat#45-0105)

### Immunizations

Mice (A/J or Balb/c, 8-10 weeks old) or rats (HSD) were immunized according to methods described in the literature. For primary immunization, animals were injected subcutaneously with recombinant antigen as an emulsion with complete Freund's adjuvant (Sigma, Mo.). Three weeks later, the antigen in an emulsion form with incomplete Freund's adjuvant (Sigma, Mo.) was injected SC to boost immune responses. A total of three boosts were given once every three weeks. A total of 25 to 50 μg of antigen was injected for each immunization. Animals with high antigen specific antibody titers were given a pre-fusion boost with recombinant antigen in PBS four days prior fusion.

### Example 1

### CD43 and IgM Immunodepletion

To enhance hybridoma technology, IgG expressing B cells (including plasmablasts, plasma cells and memory B cells) were purified from mouse spleens by depleting major irrelevant cell populations using magnetic cell separation (MACS®; Miltenyi).

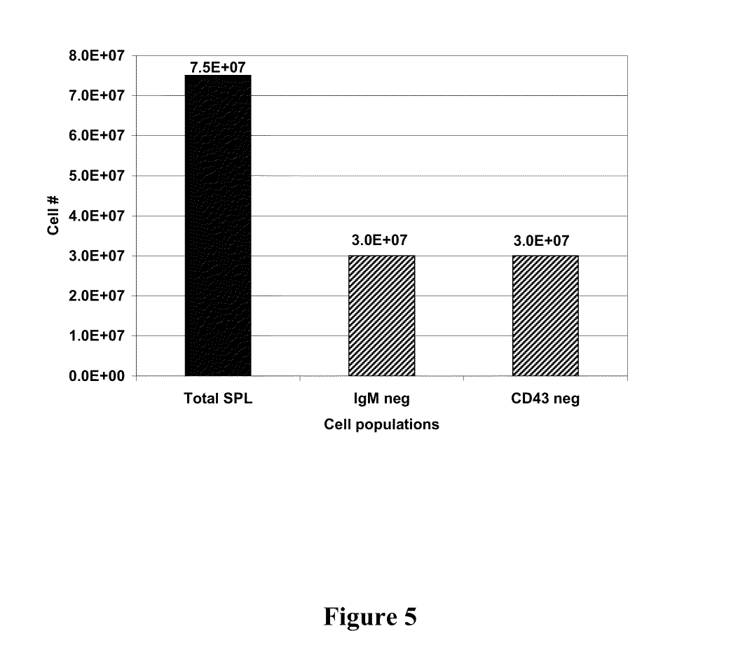

Anti-mouse IgM Microbeads were used to deplete naive B cells and anti-CD43 Microbeads were used to deplete non-B lineage cells. CD43 is a surface marker that is expressed by all hematopoietic cells, besides mature B cells (Wells S M, Kantor A B, Stall A M. CD43 (S7) expression identifies peripheral B cell subsets. J Immunol. 1994 Dec. 15; 153(12):5503-15). Both of these pre-fusion enrichment approaches resulted in approximately a 2.5-fold reduction in cell numbers, see FIG. 5. FIG. 5 displays the results of splenic cells that were isolated from a mouse immunized with a protein antigen and subjected to different sorting conditions. Depletion of IgM positive or CD43 positive cells were performed on 7.5×10⁷ splenocytes. Cell numbers before (black) and after (dashed) enrichment are shown. These numbers are in line with expected depletion of corresponding cell populations (40-60% of total splenocytes are IgM B cells and 40-60% are non-B lineage cells).

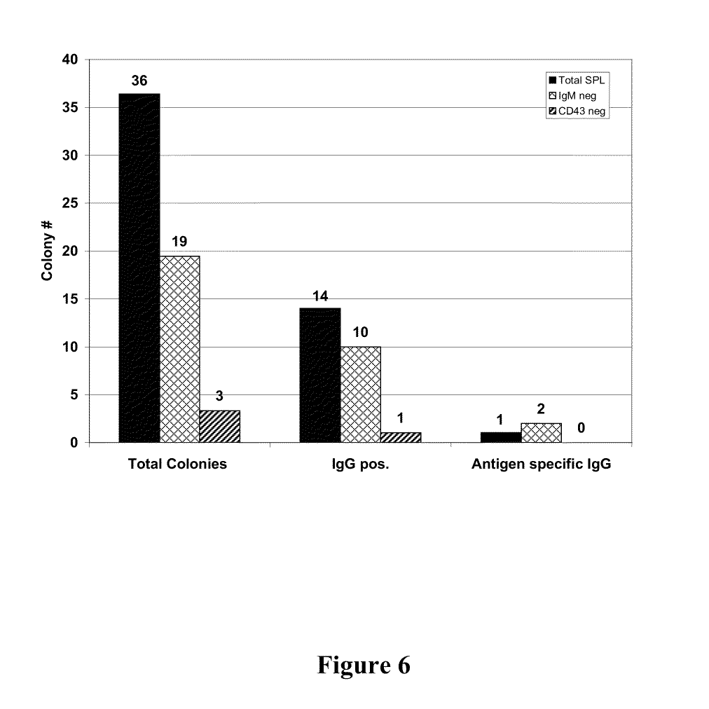

Successful fusion outcome was assessed by the total number of colony containing wells, by the number of wells containing IgG producing colonies and by the number of wells containing colonies producing target specific IgG as measured by ELISA. All of these parameters were normalized to 10⁷ initial splenocytes (before enrichment), see FIG. 6. FIG. 6 displays the results of total splenocytes, IgM negative or CD43 negative cells that were isolated as described in FIG. 5. Isolated cells were fused with myeloma cells using PEG mediated fusion and seeded at a concentration of about 10⁵ splenic cells/well.

Fusion outcome was assessed by total number of colony containing wells, number of wells containing colonies producing IgG, as well as by number of wells containing colonies producing antigen specific IgG. All numbers are normalized to 10⁷ initial splenocytes before enrichment. Data shown in FIG. 6 demonstrate an approximate 2 fold reduction in colony numbers obtained from an electrofusion done with IgM depleted cells as compared to unsorted controls. This result correlates with the reduction in cell numbers due to pre-fusion depletion of IgM negative cells and indicates that this sorting approach is compatible with an electrofusion procedure. Moreover, the numbers of wells containing IgG producing colonies, as well as the numbers of wells containing target specific IgG producing clones were comparable between IgM depleted and unsorted cells, see FIG. 6. In contrast, pre-fusion sorting using anti-CD43 microbeads, unexpectedly, resulted in very poor colony growth in two separate experiments, see FIG. 6. This provides evidence that pre-fusion enrichment by depletion IgM expressing cells can result in enhancement of hybridoma technology by reducing the total colony number without deleterious effects on the number of IgG producing clones and target specific IgG producing clones. These results also demonstrate that anti-CD43 microbeads cannot be used for pre-fusion enrichment and an alternate protocol to deplete non-B lineage cells was needed.

### Example 2

### Pan B Cell Purification and Enrichment for IgG Expressing B Cells by Immunodepletion of Mouse Splenocytes

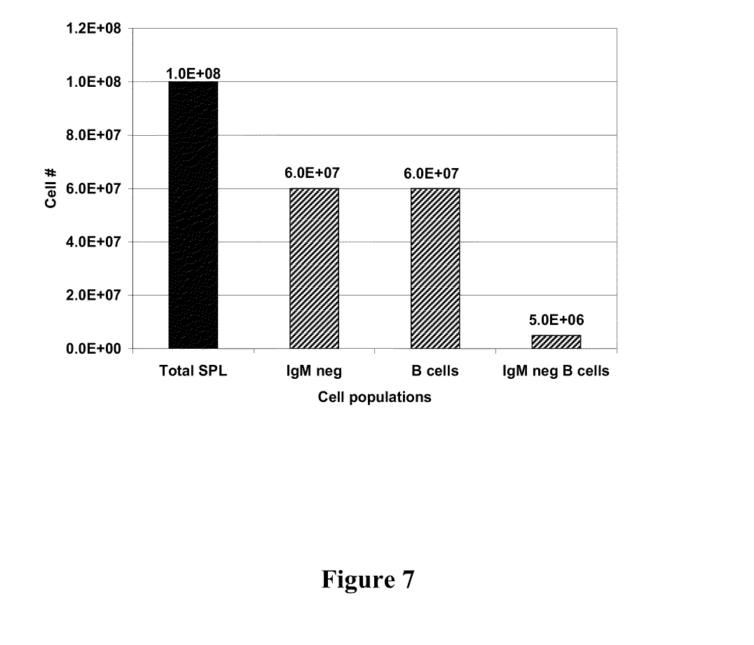

A mouse Pan B cell isolation kit was used to deplete non-B lineage cells. This kit contains a mixture of biotinylated monoclonal antibodies against surface markers such as CD4, CD90.2 (expressed on T cells); CD11c, CD49b, Gr-1 (expressed on myeloid lineage cells); and Ter-119 (expressed on red blood cells). Anti-biotin microbeads are then used as a secondary reagent to deplete cells stained with biotinylated antibodies. Pre-fusion enrichment of B cells, using this antibody cocktail results in an approximately 1.7 fold reduction in splenic cell number. Comparable enrichment has been achieved using anti-IgM microbeads. The combination of both depleting strategies resulted in an approximately 20 fold reduction in cell number, see FIG. 7. FIG. 7 displays the results of splenic cells that were isolated from four immunized mice and pooled together. Depletion of IgM positive cells is described above. B cell purification was performed using a Pan B cell isolation kit (Miltenyi) according to the manufacturer's protocol. To combine two purification protocols, cells were first stained with a mix of biotinylated mAbs, followed by incubation with anti-biotin and anti-IgM microbeads (Miltenyi). The labeled cells were depleted using LS columns (Miltenyi). All cell populations were obtained from 10⁸ initial splenocytes before enrichment. Cell numbers before (black) and after (dashed) enrichment are shown.

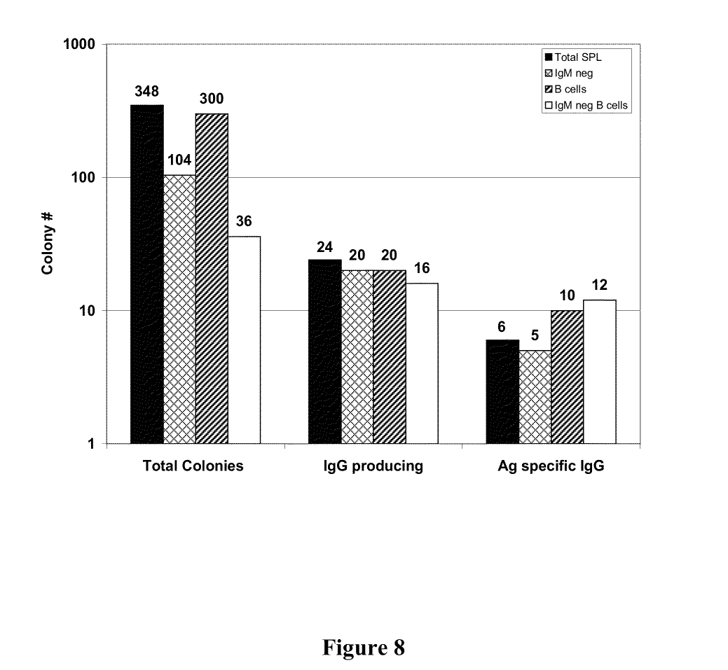

The fusion outcome of this experiment is shown in FIG. 8. FIG. 8 displays the results of total splenocytes, IgM negative, purified B cells or IgM negative B cells that were isolated as described in FIG. 7. Isolated cells were fused with myeloma cells using PEG mediated fusion and seeded at concentrations of about 1.3×10⁵ splenic cells/well. Fusion outcome was assessed by total number of colony containing wells, number of wells containing IgG producing clones, as well as by number of wells containing clones producing antigen specific IgG.

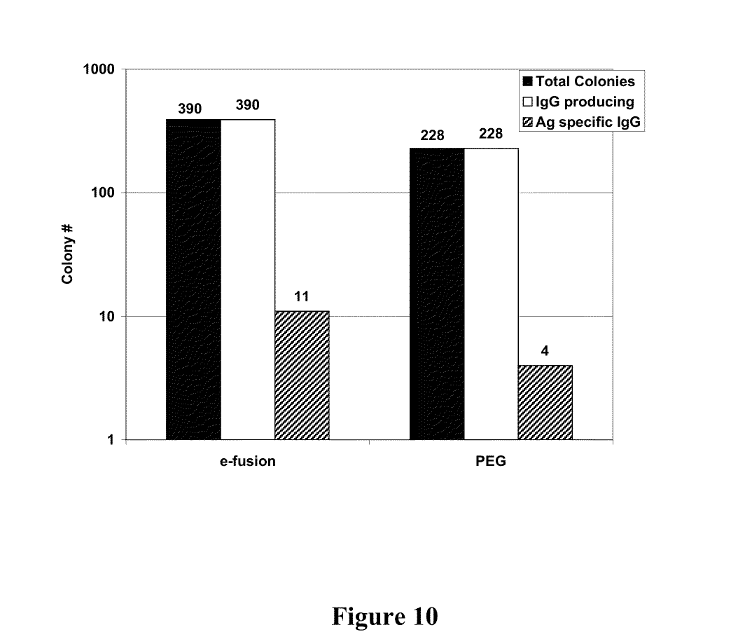

The total number of colony containing wells after depletion with anti-IgM microbeads was reduced approximately 3 fold in comparison to unsorted cells (FIG. 8) and a 20% reduction in numbers of colony containing wells was achieved after pre-fusion enrichment with a Pan B cell purification kit. The combination of both depletion strategies resulted in an approximately 10 fold reduction in numbers of colony containing wells. The number of wells containing IgG producing clones as well as the number of wells with antigen specific antigen producing clones was comparable in all four conditions. Approximately 40-100% of total colonies generated from fusions with IgM negative B cells were IgG producing, see FIG. 8 and FIG. 10. For the data shown in FIG. 10, IgM negative B cells were isolated as described in FIG. 7. Isolated cells were fused with myeloma cells using PEG mediated fusion or electrofusion. Cells were plated at concentrations of about 4×10⁴ (PEG fusion) or about 2×10⁴ (electrofusion) splenic cells/well. Fusion outcome was assessed by number of wells containing clones producing IgG, as well as by the number of wells containing clones producing antigen specific IgG. All numbers were normalized to 10⁸ initial splenocytes before enrichment.

### Electrofusion

PEG-mediated fusion often gives inconsistent results due to technical reasons. Prior to PEG-mediated fusion, splenocytes and myeloma cells are mixed together by centrifugation into a pellet with uncontrollable shape and density. Cells in the pellet form multiple contacts to an unpredicted number of neighboring cells. Slowly adding PEG and diluting it out is an undefined art, which varies enormously dependent on the technique of the operator performing the procedure.

Electrofusion has been reported to be more efficient than the PEG-mediated fusion^{3,4}. Electrofusion occurs in a controlled electric field, where the operator can establish standardized parameters and repeatable conditions. During this procedure, cells are aligned into a chain and the number of cell to cell contacts is limited. This results to higher fusion efficiency as compared to PEG-mediated fusion with significant increase in number of viable hybridoma clones. However, due to high fusion efficiency, maintenance and screening of electro-fusion outcome obtained from whole mouse spleen become very time-consuming and labor intense.

Whether pre-fusion enrichment is compatible with electrofusion was tested. Pre-fusion enrichment was performed using combined depletion with anti-IgM microbeads and a Pan B cell purification kit. Purified cells and myeloma cells were fused either by PEG or by electrofusion. The fusion outcome was evaluated and the results are shown in FIG. 10. The numbers of colonies, as well as the numbers of IgG producing clones and target specific IgG producing clones were higher in the electrofusion than in the PEG-mediated fusion by about 1.5 to about 3 fold. These data demonstrate that the combined pre-fusion enrichment protocol is compatible with electrofusion.

### Fusion Efficiency

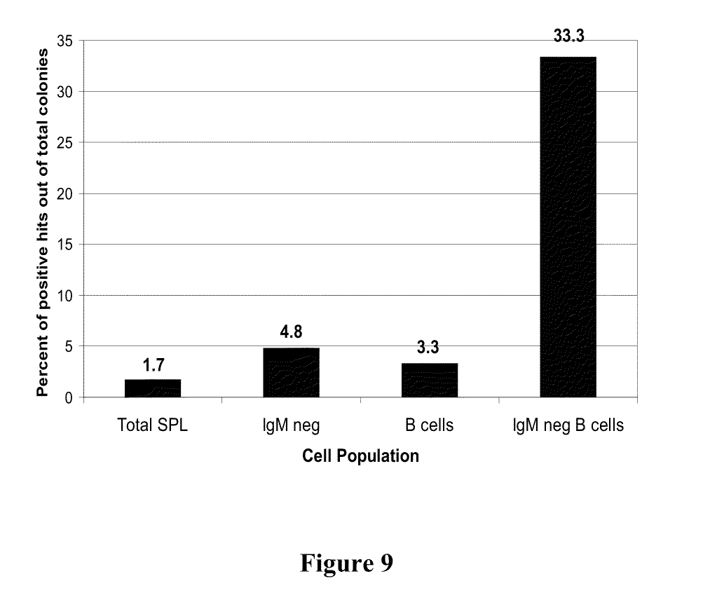

The percentage of antigen specific clones out of the total number of colony containing wells was calculated, see FIG. 9 and Table 2. FIG. 9 displays the percentage of wells containing antigen specific clones out of the total number of wells containing viable colonies for results obtained from Example 2 as depicted in FIG. 8.

The data demonstrates that pre-fusion enrichment by depletion of IgM positive cells or B cell purification leads to increase in the percentage of wells containing clones producing antigen specific IgG approximately 2.7 or 1.9 fold, respectively, while the combined sorting approach led to an increase in percentage of antigen specific clones about 4-17 fold.

Table 2 also provides information about percentage of antigen specific IgG producing hybridomas obtained from fusions of different cell populations in eight independent experiments, JS3, JS6, JS11, JS14, JS15, JS16, JS20 and JS21.

TABLE 2
Percentage of antigen specific hybridomas* obtained
from fusions of different cell populations.

| | Unsorted | Sorted cell populations | | |
|---|---|---|---|---|
| | (Total | | | IgM neg |
| | SPL) | IgM neg | B cells | B cells |
| | % antigen | % antigen | % antigen | % antigen |
| | specific | specific | specific | specific |
| Experiment ID | hybridomas | hybridomas | hybridomas | hybridomas |
| JS3 (mouse; | 12 | 24 (2x) | ND | ND |
| PEG) | | | | |
| JS6 (mouse; | 3 | 10 (3.5x) | ND | ND |
| PEG) | | | | |
| JS11 (mouse; | 0 | ND | ND | 3 |
| e-fusion) | | | | |
| JS14 (mouse; | 2 | 5 (2.5x) | 3 (1.5x) | 33 (17x) |
| PEG) | | | | |
| JS15 (mouse; | 11 | 17 (1.5x) | 18 (1.5x) | 42 (4x) |
| PEG) | | | | |
| JS16 (mouse; | 2 | 6 (3x) | 3 (1.5x) | 16 (8x) |
| PEG) | | | | |
| J520 (rat; | 1 | ND | ND | 5 (5x) |
| e-fusion) | | | | |
| JS21 (rat; | 1 | ND | ND | 4 (4x) |
| e-fusion) | | | | |

(ND—not done; *calculated as the percentage of antigen specific IgG producing clones out of the total number of viable colonies).

Mouse or rat splenocytes were isolated from immunized animals. Cell populations were isolated as described in the materials and methods and fused with NS0 myeloma cells by PEG or electrofusion (as indicated in the first column). Data from 8 independent experiments is summarized in this table.

Numbers in parentheses indicate the fold improvement over the standard fusion procedure. It is calculated as ratio of the percent of wells containing antigen specific hybridomas obtained from the fusion of sorted cells divided by percent of wells containing antigen specific hybridomas obtained from the fusion of total splenocytes in the same experiment.

### Example 3

### Immunodepletion of T- and IgM Expressing B Cells from Rat Splenocytes

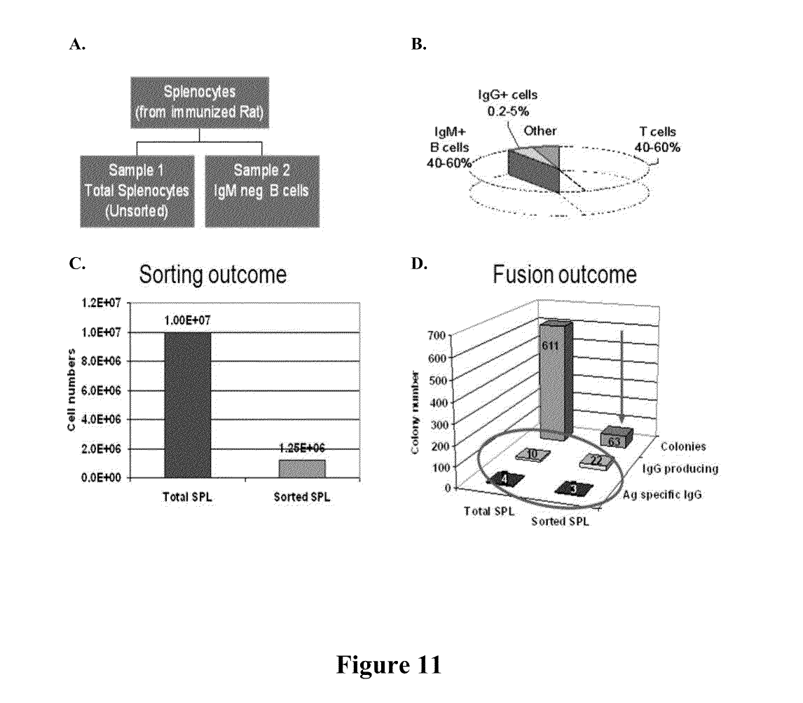

Rat splenocytes were stained with biotinylated anti-rat IgM and anti-rat TCR. Anti-biotin microbeads were then used as a secondary reagent to deplete cells stained with biotinylated antibodies. This combined depletion of T and IgM positive B cells resulted in approximately a 9 fold reduction in splenic cell number (FIG. 11C).

The fusion with NS0 myeloma cells was performed using the standard electrofusion protocol and cells were seeded at concentrations of about 1×10⁴ splenic cells/well. The fusion outcome was assessed by the total number of colonies, the number of clones producing IgG, as well as by the number of clones producing antigen specific IgG. The outcome of this experiment is shown in FIG. 11D.

The total number of colony containing wells after depletion was reduced approximately 10 fold in comparison to unsorted cells (FIG. 11D), However the number of IgG producing clones as well as the number of wells with hybridomas producing antigen specific IgG was not reduced. All numbers were normalized to 10⁷ initial splenocytes before enrichment. Most importantly, the percentage of wells containing colonies producing antigen specific IgG out of total number of colony containing wells was increased about 5-fold (see Table 2; Fusion JS20 and JS21).

### Example 4

### CpG Stimulation

Splenocytes from an immunized mouse were immunodepleted to enrich for IgG expressing B cells. Combined immunodepletion (Pan B cell purification and depletion of IgM positive B cells) is described in Example 1. After immunodepletion, splenocytes were fused with NS0 myeloma cells by electrofusion using the standard protocol, or stimulated with CpG for 18 hours prior electro-fusion. Isolated cells subject to CpG stimulation were cultured in B cell media (DMEM, 10% FCS, 1 mM sodium pyruvate, and 1× nonessential amino acids, 2 mM L-glutamine and 50 μM 2-mercaptoethanol) at 37° C. for 16 hours in the presence of 0.25 μM CpG or non-CpG oligonucleotides (InvivoGen).

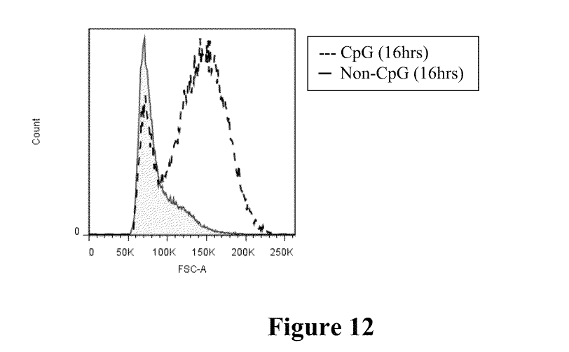

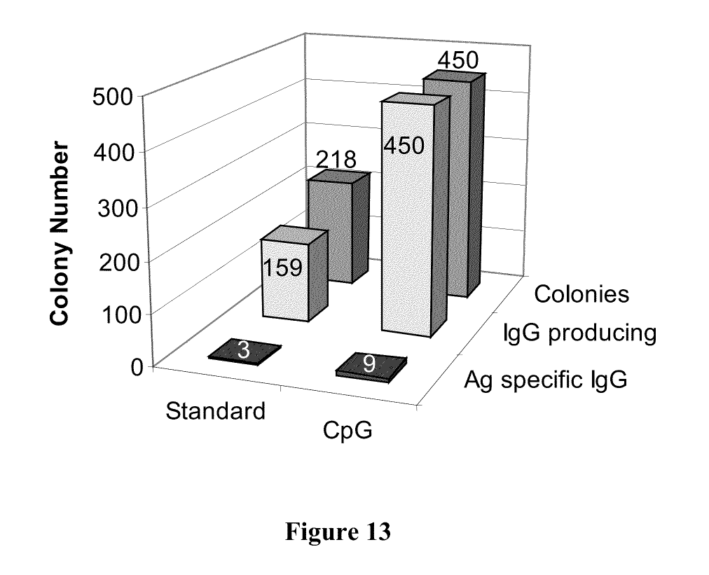

Sorted cells were stimulated either with CpG or Non-CpG oligonucleotides for 16 hours, and analyzed by flow cytometry. CpG stimulation led to the increase in cell size of sorted cells as measured by forward scatter (correlative of cell size) (FIG. 12), indicative of blastogenesis. The data in FIGS. 12 and 13 as well as Table 3, demonstrate that enlarged cell size correlates with improved fusion efficiency over the standard protocol, as measured by about a 2.5-fold increase in total colony number, number of IgG producing colonies and number of colonies producing antigen specific IgG (FIG. 12).

TABLE 3
CpG stimulation of sorted cells increases efficiency of electrofusion

| | | | | Ag |
|---|---|---|---|---|
| | Pre-fusion | Number of sorted | Total | specific IgG |
| Experiment ID | Stimulus | splenocytes fused | Colonies | colonies |
| SJ6 | No stimuli | 8.6 × 10⁶ | 1440 | 0 |
| | CpG | 8.6 × 10⁶ | 5760* | 4 |
| | Non-CpG | 8.6 × 10⁶ | 1440 | 0 |
| SJ8 | No stimuli | 1.3 × 10⁷ | 218 | 3 |
| | CpG | 1.3 × 10⁷ | 450 | 9 |
| | Non-CpG | 1.3 × 10⁷ | 104 | 1 |
| SJ9 | No stimuli | 1 × 10⁷ | 140 | 14 |
| | CpG | 1 × 10⁷ | 894* | 65 |
| | Non-CpG | 1 × 10⁷ | 144 | 2 |
| SJ10 | No stimuli | 5 × 10⁶ | 1871* | 42 |
| | CpG | 5 × 10⁶ | 3464* | 46 |
| | Non-CpG | 5 × 10⁶ | 277 | 10 |

*An estimate of total colony number, based on an observed average of 2-3 colonies per well.

To determine whether the increase in fusion efficiency was associated with an increase in frequency of duplicate clones, antibodies were purified from fusion SJ9 and it was determined if CpG stimulation increased the number of identical/duplicated antigen specific IgG producing hybridomas compared to the no-stimulation controls by mass spectrometry (Table 4).

TABLE 4
Pre-fusion CpG stimulation does not result in
increased frequency of duplicate hybridoma clones:

| | | |
|---|---|---|
| SJ9 Clone Name | Heavy Chain (da) | Light Chain (da) |

No Stimulation

| | | |
|---|---|---|
| 5G1 | 49585 | 24113 |
| 9F9 | 49800 | 24031 |
| 9H4 | 52550 | 23423, |
| | | 23528 |
| 1197 | 50204 | 24303 |
| 12A11 | 50883 | 24154 |
| 14G11 | 50663 | 23293 |
| 16D3 | 49863 | 23907 |
| 17G1 | 50137 | 24103 |
| 17G3 | 50656 | 23307 |
| 20A4 | 50832 | 23622 |
| 20E12 | 50886 | 23642 |
| 20G8 | 51204 | 24393 |

CpG Stimulation

| | | |
|---|---|---|
| 21A5.1F4 | 50219 | 24275 |
| 21C8.1C12 | 50163 | 24117 |
| 22A12.1G2* | 50274 | 24288 |
| 22B1.1H8 | 49225 | 24161 |
| 22D5.1G1 | 49600 | 24046 |
| 22E10.1E1 | 50654 | 23904 |
| 22E12.1F4 | 50742 | 23904 |
| 23E4.1E1 | 50121 | 24168 |
| 24A11.1H11* | 50273 | 24288 |
| 24B9.1F7 | 50637 | 23930 |
| 24C11.1C12 | 50220 | 24345 |
| 24H10.1E6 | 50952 | 24251 |
| 25E1.1E4 | 49571 | 24118 |
| 25G9.1C11** | 50610 | 23930 |
| 26D7.1C7 | 51057 | 23389 |
| 26G4.1B12 | 50137 | 24093 |
| 28D1.1A2 | 50708 | 23845 |
| 30B11.1C4 | 51969, | 24119 |
| | 51951 | |

Non-CpG Stimulation

| | | |
|---|---|---|
| 38D10** | 50610 | 23930 |

*Repeated Antibody pair 1
**Repeated Antibody pair 2

The data demonstrate that, of the 31 antibodies, 27 were unique as determined by the unique molecular weights of the heavy and the light chains. Two pairs of antibodies have identical heavy and light chains. One pair of repeated clones (24A11.1H11 and 22A12.1G2) originated from the CpG stimulated cells, while another pair of repeated clones (25G9.1C11 and 38D10), originated from either CpG or Non-CpG stimulated cells. These data suggest that pre-fusion CpG stimulation does not result in a significant increase in frequency of repeated clones.

### Example 5

### Micro Parallel Hybridoma (MPH) Screening

An immunodepleted B-cell library (e.g., 1-3 million B-cells) of the invention may be arrayed in a multi-well (e.g., 2000×2000) array wherein each well of the array is of sufficient size (e.g., 30 microns) to accommodate the ˜15 micron diameter (˜2 pl volume) of a single B-cell. A separate array of 1-3 million myeloma cells is also provided wherein each well is able to accommodate the ˜20 micron diameter or ˜4 pl volume of a single myeloma cell. The two arrays may then be positioned such that a single well of the B-cell array library is capable of aligning with a single well of the myeloma array library. The two aligned array plates may connected to electrodes and supplied with electrical current such that a single myeloma cell in each well of the myeloma library is capable of pairing and fusing with a single B-cell from a corresponding aligned well of the B-cell library. The fused cells may then be cultured in the array plate for subsequent antibody detection and/or isolation.

The advantage of this “micro-parallel” screening approach is that the precise pairing of myeloma and B-cells and the reduced voltage requirements due to reduced spacing (<100 um) between electrodes allow for increased fusion efficiency and increased hybridoma recovery. Moreover, the microfluidic volumes employed permit the assaying for antibodies expressed at much lower concentrations. Finally, the massive parallelization of this approach allows for significant reduction in timelines for antibody generation and screening for antibodies of desired binding characteristics.

### Example 6

### Enrichment for IgG Expressing B Cells by Immunodepletion of Mouse Splenocytes Using RoboSep

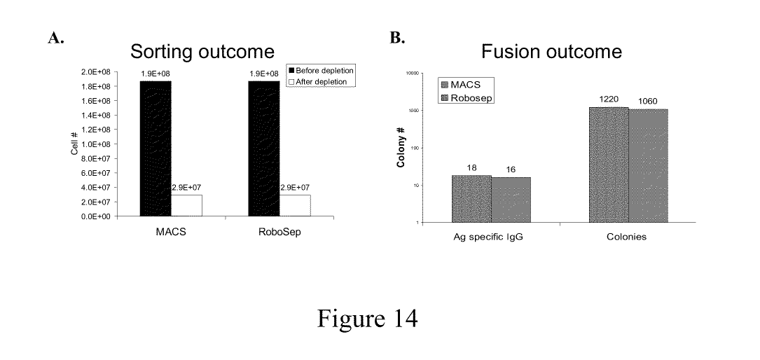

Enrichment for IgG expressing B cells by immunodepletion of mouse splenocytes was performed using MACS (Miltenyi) (as described in Example 2) or using fully automated cell isolation system (RoboSep; StemCell Technologies). For immunodepletion by RoboSep biotinylated anti-IgM antibody was added to a mixture of biotinylated monoclonal antibodies against surface markers such as CD4, CD8 (expressed on T cells); CD11b, CD49b, Gr-1 (expressed on myeloid lineage cells); and Ter-119 (expressed on red blood cells). Both depleting systems resulted in an approximately 66 percent reduction in total cell numbers, see FIG. 14A. Cell numbers before (black) and after (white) enrichment are shown.

Isolated cells were fused with myeloma cells using e-fusion and seeded at concentrations of about 1×10⁴ splenic cells/well. Fusion outcome was assessed by total number of colonies, and by number of clones producing antigen specific IgG. The fusion outcome of this experiment is shown in FIG. 14B. Fusions of cells enriched by immunodepletion using Miltenyi or RoboSep systems led to comparable numbers of colony containing wells and number of hybridomas producing antigen specific IgG.

### Example 7

### Enrichment for IgG Expressing Cells Isolated from Rat Lymph Nodes

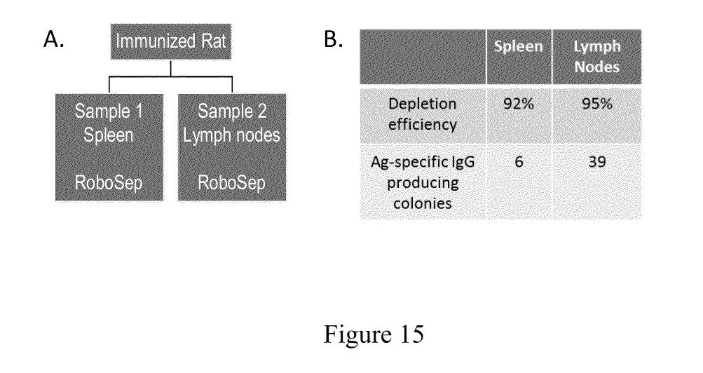

Enrichment for IgG expressing B cells by immunodepletion of lymphocytes isolated from rat spleen or rat lymph nodes was performed using RoboSep (as described in example 2). Percent of cells recovered from depletion of lymph node cells was comparable to those of splenic cells (FIG. 15B). Moreover, electrofusion of enriched cells with myelomas resulted to viable productive hybridoma lines independent of the original source (spleen or lymph nodes) (FIG. 15B).

1. A method of generating a hybridoma comprising
a) providing spleen or lymph node cells from an animal and myeloma cells;
b) isolating IgM negative B cells from the spleen or lymph node cells; and
c) fusing the isolated IgM negative B cells and the myeloma cells;
thereby generating a hybridoma.

2. The method of claim 1, wherein the murine spleen or lymph node cells are isolated from animals immunized with soluble recombinant proteins, cell lines, purified human or humanized antibodies.

3. The method of claim 2, wherein the hybridoma produce antibodies that specifically bind to soluble recombinant proteins, cell-surface glycoproteins or anti-idiotypic antibodies.

4. The method of claim 3, wherein the anti-idiotypic antibodies specifically bind to variable domains of human or humanized antibodies.

5. The method of claim 1, wherein the fusion is electrofusion.

6. The method of claim 5, further comprising CpG stimulation of the isolated IgM negative B cells prior to fusion.

7. The method of claim 6, wherein the CpG stimulation is performed 1-24 hours prior to fusion.

8. The method of claim 1, wherein the IgM negative B cells are isolated by immunodepleting IgM positive cells and non-B cells.

9. The method of claim 8, wherein the immunodepletion is performed by depleting the spleen or lymph node cells of IgM positive cells.

10. The method of claim 8, wherein the immunodepletion is performed by depleting the spleen or lymph node cells of one or more of TCR, CD4, CD90.2, CD11c, CD49b, Gr-1 or Ter-119 positive cells.

11. The method of claim 8, wherein the immunodepletion is performed by depleting the spleen or lymph node cells of IgM positive cells and one or more of TCR, CD4, CD90.2, CD11c, CD49b, Gr-1 or Ter-119 positive cells.

12. The method of claim 8, wherein the immunodepletion is performed by depleting the spleen or lymph node cells of non-B cells.

13. The method of claim 12, wherein the immunodepletion is performed by depleting the spleen or lymph node cells of IgM positive cells.

14. The method of claim 1, wherein the fusion of the isolated IgM negative B cells and the myeloma cells is performed by electrofusion.

15. The method of claim 1, further comprising screening the hybridomas for antigen specific IgG.

16. The method of claim 12, wherein the screening is performed using ELISA, flow cytometry or imaging.

17. The method of claim 1, wherein the spleen or lymph node cells are isolated from a vertebrate animal selected from the group consisting of rodent, rabbit, goat, sheep, donkey, llama, camel, monkey, chimpanzee and human.

18. The method of claim 1, wherein the splenocyte is isolated from a rodent, wherein the rodent is a mouse or rat.

19. A method of isolating IgM negative B cells from murine spleen or lymph node cells comprising
a) providing murine spleen or lymph node cells; and
b) immunodepleting IgM positive cells and non-B cells,
thereby isolating IgM negative B cells.

20. The method of claim 19, wherein the IgM negative B cells are also TCR, CD4, CD90.2, CD11c, CD49b, Gr-1 or Ter-119 negative cells.

21. The method of claim 19 further comprising fusing the isolated IgM negative B cells and murine myeloma cells thereby generating hybridoma.

22. The method of claim 19, further comprising screening the hybridomas for antigen specific IgG.

23. A monoclonal antibody produced by the method of claim 1.

24. A method for treating an antigen-related disease in a subject, comprising administering the antibody of claim 23 to the subject.

25. An isolated population of B cells, wherein the B cells are IgM negative.

26. The population of claim 25, wherein the B cells are TCR, CD4, CD90.2, CD11c, CD49b, Gr-1 or Ter-119 negative.

27. The population of claim 25, wherein at least 25% of the B cells express IgG.

28. The population of claim 25, wherein at least 50% of the B cells express IgG.
